(19) United States
(12) Patent Application Publication
(10) Pub. No.: US 2008/0262416 A1
(43) Pub. Date: Oct. 23, 2008

(54) Microneedle Arrays and Methods of Preparing Same

(76) Inventors: Daniel C. Duan, St. Paul, MN (US); Peter R. Johnson, Eagan, MN (US)

Correspondence Address: 3M INNOVATIVE PROPERTIES COMPANY, PO BOX 33427, ST. PAUL, MN, 55133-3427, US

(21) Appl. No.: 12/089,551

(22) PCT Filed: Nov. 17, 2006
(86) PCT No.: PCT/US2006/044551

§ 371 Date: Apr. 8, 2008

Related U.S. Application Data
(60) Provisional application No. 60/747,610, filed on May 18, 2006.

(30) Foreign Application Priority Data
Nov. 18, 2005 (US) PCT US2020 05 041

(51) Int. Cl. A61M 31/00; A61M 37/00; B05D 5/00

(52) U.S. Cl. 604/46; 427/2.1

(57) ABSTRACT

A microneedle array having a substrate and a plurality of microneedles extending out from the substrate with a multi-phase matrix coating on at least a portion of the microneedle surface of the microneedle array. The multi-phase matrix coating comprises an active substance and has a first solid phase and a liquid phase. The first solid phase comprises a water-soluble polymer. Also, a method of providing an active substance- containing matrix coating on a microneedle array in which a water-soluble polymer is applied to the microneedle surface of the array to form a dried coating of water-soluble polymer. A coating solution comprising an active substance, a liquid capable of phase separating from the water-soluble polymer, and a carrier fluid is prepared and applied to the dried coating of water-soluble polymer. At least a portion of the carrier fluid is removed from the array.

### CROSS-REFERENCE TO RELATED APPLICATIONS

This application claims priority to International Patent Application Serial No. US2005/041858, filed on Nov. 18, 2005, and to U.S. Provisional Application Ser. No. 60/747618, filed on May 18, 2006, both of which are incorporated herein by reference.

### Field

The present invention relates to microneedle arrays and methods of preparing the same, and in particular to microneedle arrays where a drug, such as a vaccine, is coated on the microneedles.

### Background

Devices including arrays of many small piercing structures, sometimes referred to as microneedles, microblades, or micro-pins, have been disclosed for use in connection with the delivery of drugs and other substances through the skin and other surfaces. The devices are typically pressed or driven against the skin in an effort to pierce the stratum corneum such that the drugs and other substances can pass through that layer and into the tissues below.

Some microneedle devices have a fluid reservoir and conduits through which a therapeutic substance may be delivered into or through the skin. Others have a coating on the surface of the microneedles that releases into the target tissue after penetration.

Summary of the Invention It has been found, however, that the ability to provide a consistent coating in one or more desired locations on the microneedle array is an important feature for a microneedle device having an active substance-containing matrix coating disposed on the surface of the microneedle array. Although there are numerous well known methods for providing dried coatings on generally flat surfaces, coating of a microneedle array provides a challenge due to the high surface irregularity inherent in the array design.

In a first aspect, the present invention provides a microneedle array comprising a substrate and a plurality of microneedles extending out from the substrate with a multi-phase matrix coating on at least a portion of the microneedle surface. The multi-phase matrix coating comprises an active substance and has a first solid phase and a liquid phase. The first solid phase comprises a water-soluble polymer,

In a second aspect, the present invention provides a method of providing an active substance-containing matrix coating on a microneedle array. A microneedle array is provided having a substrate and a plurality of microneedles extending out from the substrate. A water-soluble polymer is applied to the microneedle surface to form a dried coating of water-soluble polymer on at least a portion of the microneedles. A coating solution comprising an active substance, a liquid capable of phase separating from the water-soluble polymer, and a carrier fluid is prepared and applied to the dried coating of water-soluble polymer. At least a portion of the carrier fluid is removed from the array to provide the active substance-containing matrix coating on a microneedle array.

In certain embodiments, the present invention may provide one or more of the following benefits: protection of active substance(s) from contact, and thus possible irreversible adhesion, with a microneedle array; protection of active substance(s) from accidental removal from a microneedle array; relative ease of removal of active substance(s) from a microneedle array under ordinary conditions of usage; and efficient placement of active substance(s) at or near the tips of the microneedles.

The invention will be further understood by those skilled in the art upon consideration of the remainder of the disclosure, including the Detailed Description and the appended claims.

As used herein, certain terms will be understood to have the meaning set forth below:

“Array” refers to the medical devices described herein that include one or more structures capable of piercing the stratum corneum to facilitate the transdermal delivery of therapeutic agents or the sampling of fluids through or to the skin.

“Microstructure,” “microneedle” or “microarray” refers to the specific microscopic structures associated with the array that are capable of piercing the stratum corneum to facilitate the transdermal delivery of therapeutic agents or the sampling of fluids through the skin. By way of example, microstructures can include needle or needle-like structures as well as other structures capable of piercing the stratum corneum.

The features and advantages of the present invention will be understood upon consideration of the detailed description of the preferred embodiment as well as the appended claims. These and other features and advantages of the invention may be described below in connection with various illustrative embodiments of the invention. The above summary of the present invention is not intended to describe each disclosed embodiment or every implementation of the present invention. The Figures and the detailed description that follow more particularly exemplify illustrative embodiments.

### BRIEF DESCRIPTION OF THE DRAWINGS

Preferred embodiments of the invention will now be described in greater detail below with reference to the attached drawings, wherein:

FIG. 1A is a microphotograph of a multi-phase matrix coating.

FIG. 1B is a microphotograph of a porous solid coating remaining after the liquid was removed from the multi-phase matrix coating in FIG. 1A.

FIGS. 2A-5A are microphotographs of other multi-phase matrix coatings.

FIGS. 2B-5B are microphotographs of porous solid coatings remaining after the liquid was removed from the multi-phase matrix coatings of FIGS. 2A-5A, respectively.

### DETAILED DESCRIPTION

One embodiment of the present invention comprises a microneedle array having a substrate and a plurality of microneedles extending out from the substrate with a multi-phase matrix coating on at least a portion of the microneedle surface of the microneedle array. The multi-phase matrix coating comprises an active substance and has a first solid phase and a liquid phase. The first solid phase comprises a water-soluble polymer.

Microneedle arrays useful in the various embodiments of the invention may comprise any of a variety of configurations, such as those described in the following patents and patent applications, the disclosures of which are herein incorporated by reference. One embodiment for the microneedle arrays comprises the structures disclosed in United States Patent Application Publication No. 2003/0045837. The disclosed microstructures in the aforementioned patent application are in the form of microneedles having tapered structures that include at least one channel formed in the outside surface of each microneedle. The microneedles may have bases that are elongated in one direction. The channels in microneedles with elongated bases may extend from one of the ends of the elongated bases towards the tips of the microneedles. The channels formed along the sides of the microneedles may optionally be terminated short of the tips of the microneedles. The microneedle arrays may also include conduit structures formed on the surface of the substrate on which the microneedle array is located. The channels in the microneedles may be in fluid communication with the conduit structures. Another embodiment for the microneedle arrays comprises the structures disclosed U.S. Patent Application Publication No. 2005/0261631, which describes microneedles having a truncated tapered shape and a controlled aspect ratio. Still another embodiment for the microneedle arrays comprises the structures disclosed in U.S. Pat. No. 6,091,975 (Daddona, et al.) which describes blade-like microprotrusions for piercing the skin. Still another embodiment for the microneedle arrays comprises the structures disclosed in U.S. Pat. No. 6,313,612 (Sherman, et al.) which describes tapered structures having a hollow central channel. Still another embodiment for the microneedle arrays comprises the structures disclosed in U.S. Pat. No. 6,379,324 (Gartstein, et al.) which describes hollow microneedles having at least one longitudinal blade at the top surface of the tip of the microneedle.

The microneedles are typically less than 1000 microns in height, often less than 500 microns in height, and sometimes less than 250 microns in height. The microneedles are typically more than 5 microns in height, often more than 25 microns in height, and sometimes more than 100 microns in height. The microneedles may be characterized by an aspect ratio. As used herein, the term “aspect ratio” is the ratio of the height of the microneedle (above the surface surrounding the base of the microneedle) to the maximum base dimension, that is, the longest straight-line dimension that the base occupies (on the surface occupied by the base of the microneedle). In the case of a pyramidal microneedle with a rectangular base, the maximum base dimension would be the diagonal line connecting opposed corners across the base. Microneedles typically have an aspect ratio of between about 2:1 to about 5:1 and sometimes between about 2.5:1 to about 4:1.

The microneedles may be made of any suitable material, such as polymers, metals, or ceramics. Among polymeric materials, it may be preferred that the microneedles be manufactured of thermoplastic polymeric materials. Suitable polymeric materials for the microneedles of the present invention may include, but are not limited to: polyacrylonitrile, polyacrylonitrile-butadiene, polybutadiene-styrenes, polyphenyl sulfides, polycarbonates, polypropylenes, acetals, acrylics, polyetherimides, polybutylene terephthalates, and polyethylene terephthalates. Polymeric microneedles may be manufactured of a single polymer or a mixture/blend of two or more polymers. In one embodiment, the polymeric material is water-insoluble.

The microneedle arrays are generally characterized as having a substrate from which a plurality of microneedles protrudes. The area of the substrate from which the microneedles protrude may have any suitable size, but will typically have a planar area of between about 0.5 cm² and about 10 cm².

A multi-phase matrix coating is present on at least a portion of the microneedle surface of the microneedle array. The multi-phase matrix coating may be substantially evenly distributed on the microneedle surface of the microneedle array. In another embodiment, the multi-phase matrix coating is preferentially present on the substrate. In another embodiment, the multi-phase matrix coating is preferentially present on the microneedles. By preferentially present it is meant that the amount of multi-phase matrix coating per unit surface area will be greater in one area than in another. In one embodiment, the multi-phase matrix coating is preferentially present on or near the tips of the microneedles. In some cases more than 20%, sometimes more than 50%, and occasionally more than 75% of the multi-phase matrix coating by weight is present on the microneedles. In some cases the multi-phase matrix coating preferentially resides on the upper half of the microneedles, that is, the portion of the microneedles away from the substrate. In one embodiment, substantially no multi-phase matrix coating is present on the substrate, that is, substantially all of the multi-phase matrix coating is present on the microneedles. In one embodiment, substantially all of the multi-phase matrix coating is present on the upper half of the microneedles. By substantially all, it should be understood that insignificant amounts of multi-phase matrix coating, for example less than about 5% by weight, preferably less than about 1% by weight of the multi-phase matrix coating are present on the lower half of the microneedles and the substrate. The thickness of the multi-phase matrix coating may vary depending on the location on the microneedle array and the intended application use for the coated microneedle array. Typical thicknesses of the multi-phase matrix coating are less than 50 microns, often less than 20 microns and sometimes less than 10 microns. It may be desirable for the coating thickness to be smaller near the tip of the microneedle so as not to interfere with the ability of the microneedle to effectively pierce into the skin.

The multi-phase matrix coating comprises a first solid phase comprising a water-soluble polymer. Examples of suitable polymers include polyvinylpyrrolidone, polyvinyl alcohol, polyethylene glycol, poly(ethyl oxazoline), polyacrylamide, poly(N-vinyl formamide), poly(N-vinyl acetamide), poly(N,N-dimethyl acrylamide), polyvinyl oxazolidone, poly(ethylene oxide), poly(acrylic acid) and its partial or complete salts, cellulose derivatives, such as ethyl cellulose, hydroxyethyl cellulose, hydroxypropyl cellulose, and hydroxypropyl methyl cellulose, guar gum, xanthan gum, agarose, and copolymers and/or mixtures thereof. In one embodiment the water soluble polymer is polyvinyl alcohol. In one embodiment the water soluble polymer is polyvinylpyrrolidone. For purposes of the present invention it is sufficient that some portion of the polymer be water-soluble under readily achievable conditions. So, for example, it may be necessary to heat certain polymers to allow them to dissolve in water. Likewise it should be appreciated that these polymers may contain high-molecular weight fractions or be partially cross-linked so that some portion of the polymer may not be able to dissolve in water.

The multi-phase matrix coating further comprises a liquid phase. In its most general sense a liquid may be considered a material whose molecules are free to move past one another (i.e., not solid) while remaining in sliding contact with one another (i.e., not gaseous). Such a definition, however, may potentially apply to any amorphous material. For example, amorphous high-molecular weight polymers may flow over very long time scales (e.g., months, years, etc.) if held at temperatures above their glass transition temperature but below their molten temperature, but will not generally be considered to be liquid. For purposes of the present invention, a liquid is considered to be a material where the molecules can readily undergo flow during a time scale relevant to formation of the multi-phase matrix coating, which is typically on the order of less than one minute to 30 minutes, and under conditions relevant to formation and use of the multi-phase matrix coatings, such as room temperature and pressure. Determination of flow may be performed by any suitable test method, such as ASTM D 4359-90(2000)el, “Standard Test Method for Determining Whether a Material is a Liquid or a Solid”. Typical liquid phases will have a viscosity of between about 10⁻⁴ and 100 Pa·s, often between about 10⁻³ and 10 Pa·s, and sometimes between about 0.1 and 5 Pa·s. It should be appreciated, however, that the liquid phase may be held in place in the multi-phase matrix and/or on the microneedle surface by capillary or surface tension forces, since these forces may be sufficient to prevent bulk flow of microscopic layers or channels of liquid. Even in the absence of bulk flow, however, the molecules within the microscopic layers or channels of liquid will still be able to flow with respect to each other.

Suitable liquids include those capable of phase separating from the water-soluble polymer. In one embodiment the liquid phase comprises a surfactant. Examples of suitable surfactants include polyoxyethylene sorbitan esters, such as polyoxyethylene (20) sorbitan monooleate (available as TWEEN 80), polyoxyethylene (20) sorbitan monostearate (available as TWEEN 60), polyoxyethylene (5) sorbitan monooleate (available as TWEEN 81), and polyoxyethylene (20) sorbitan monolaurate (available as TWEEN 20); sorbitan esters, such as sorbitan monolaurate (available as SPAN 20), sorbitan monooleate (available as SPAN 80), and sorbitan trioleate (available as SPAN 85); mono and diglycerides, such as glycerol monooleate; and mixtures thereof. Other suitable liquids include glycerol and propylene glycol. In one embodiment, the liquid phase comprises an oily liquid having a viscosity greater than that of water.

In one embodiment, the liquid phase is a continuous phase and the first solid phase is present as discrete particles suspended in the liquid phase and/or contacting the microneedle array surface. Such discrete solid particles may take any shape, such as spherical, needle-shaped, plate-like, and the like. The solid particles may have relatively smooth surfaces, irregular or rough surfaces, or some combination thereof and may be either solid or porous.

In another embodiment, the multi-phase matrix coating is bicontinuous, that is, the liquid phase is a continuous phase and the first solid phase is present as an interlocking continuous phase within the liquid phase. In still another embodiment, the liquid phase is present as discrete domains. For example, the liquid phase may be present as droplets suspended within a continuous, first solid phase, or as droplets contained within depressions within the first solid phase. Presence of a continuous solid phase may be beneficial in that the liquid phase will be generally contained within the solid phase and thus potentially protected from accidental removal from the microneedle array.

The weight ratio of first solid phase to liquid phase is typically greater than about 5:95, often greater than about 10:90, and sometimes greater than about 20:80. The weight ratio of first solid phase to liquid phase is typically less than about 90: 10, often less than about 80:20, and sometimes less than about 50:50.

The matrix coating on the microneedle array comprises one or more of a biologically active material, a pharmaceutically effective substance, and a therapeutically active substance, which are collectively referred to throughout as active substances. In one embodiment, microneedle devices suitable for use in the present invention may be used to deliver drugs (including any pharmacological agent or agents) through the skin in a variation on transdermal delivery, or to the skin for intradermal or topical treatment, such as vaccination. In one aspect, drugs that are of a large molecular weight may be delivered transdermally. Increasing molecular weight of a drug typically causes a decrease in unassisted transdermal delivery. Microneedle arrays suitable for use in the present invention have utility for the delivery of large molecules that are ordinarily difficult to deliver by passive transdermal delivery. Examples of such large molecules include proteins, peptides, nucleotide sequences, monoclonal antibodies, DNA vaccines, polysaccharides, such as heparin, and antibiotics, such as ceftriaxone.

In another aspect, microneedle arrays suitable for use in the present invention may have utility for enhancing or allowing transdermal delivery of small molecules that are otherwise difficult or impossible to deliver by passive transdermal delivery. Examples of such molecules include salt forms; ionic molecules, such as bisphosphonates, including sodium alendronate or pamedronate; and molecules with physicochemical properties that are not conducive to passive transdermal delivery.

In another aspect, microneedle arrays suitable for use in the present invention may have utility for enhancing delivery of molecules to the skin, such as in dermatological treatments, vaccine delivery, or in enhancing immune response of vaccine adjuvants. Examples of suitable vaccines include flu vaccine, Lyme disease vaccine, rabies vaccine, measles vaccine, mumps vaccine, chicken pox vaccine, small pox vaccine, hepatitis vaccine, pertussis vaccine, rubella vaccine, diphtheria vaccine, encephalitis vaccine, yellow fever vaccine, recombinant protein vaccine, DNA vaccine, polio vaccine, therapeutic cancer vaccine, herpes vaccine, pneumococcal vaccine, meningitis vaccine, whooping cough vaccine, tetanus vaccine, typhoid fever vaccine, cholera vaccine, tuberculosis vaccine, and combinations thereof. The term “vaccine” thus includes, without limitation, antigens in the forms of proteins, polysaccarides, oligosaccarides, or weakened or killed viruses. Additional examples of suitable vaccines and vaccine adjuvants are described in United States Patent Application Publication No. 2004/0049150, the disclosure of which is hereby incorporated by reference.

In one embodiment, the present invention is a method of applying an active substance-containing matrix coating to the surface of a microneedle array. A microneedle array having a microneedle surface comprising a substrate and a plurality of microneedles is provided. A water-soluble polymer is applied to the microneedle surface to form a dried coating of water-soluble polymer on at least a portion of the microneedles surface. In one embodiment, the water-soluble polymer coating may be sterilized, for example, by exposure to gamma radiation or ethylene oxide gas.

For example, a thin coating of water-soluble polymer may be applied to the entire surface of the array prior to application of the coating solution, described in more detail below. Such a coating of water-soluble polymer may alter the hydrophilicity or hydrophobicity of the array and thereby affect the ability of the coating solution to wet the array. Such a coating of water-soluble polymer may also be partially or totally miscible with the coating solution, so that the water-soluble polymer is at least partially taken up into the coating solution before the carrier solvent completely evaporates, thereby leaving a mixture of the water-soluble polymer and the coating material subsequently applied. Examples of suitable water-soluble polymers are described above. Such a polymer coating may be prepared on the array by applying a polymer solution (e.g., an aqueous or ethanolic solution) to the array and allowing the solvent to evaporate, thereby leaving a dried polymer coating behind. Alternatively, the coating of water-soluble polymer may be directly applied as a solid material, such as through use of heat or plasma deposition. In certain embodiments, it may be desirable to add a surfactant to the polymer solution to aid in spreading the polymer across the entire surface of the microneedle array. The surfactant used to aid spreading of the polymer solution may be the same or different from the surfactant used in the coating solution that is subsequently applied to the polymer coating. In some instances, the surfactant may also aid in spreading of the coating solution that is subsequently applied to the dried coating of water-soluble polymer. Other additives or excipients may also be included in the coating of water-soluble polymer.

A coating solution comprising a carrier fluid and one or more active substances is subsequently prepared and applied to the dried coating of water-soluble polymer. In one embodiment, the coating solution is allowed to remain in contact with the dried coating of water-soluble polymer for a length of time sufficient to allow at least a portion of the dried coating of water-soluble polymer to be dissolved, at which time the carrier fluid is removed thus leaving behind any non-volatile components, such as active substances and other inactive pharmaceutical excipients.

The coating solution comprises an active substance, a liquid, and a carrier fluid or solvent. The carrier fluid or solvent should be selected so that it may dissolve or disperse the active substance. In one embodiment, the carrier fluid is selected so that it may dissolve at least a portion of the initially formed dried coating of water-soluble polymer. Dispersed material may be in the form of a suspension, that is, as particles dispersed or suspended in a carrier fluid or solvent. Examples of suitable carrier fluids or solvents include water, ethanol, methanol, isopropanol, ethyl acetate, hexane, and heptane. Water is particularly preferred as a carrier fluid. The coating solution may contain additional excipients such as viscosity modifiers, stabilizers, pH modifiers, and other additives. Examples of suitable additional excipients include sugars, such as sucrose, trehalose, raffinose, and lactose; proteins, such as ovalbumin; and salts, such as monosodium citrate, disodium citrate, trisodium citrate, monopotassium citrate, dipotassium citrate, and tripotassium citrate. In one embodiment, the coating solution may contain a water-soluble polymer, which may or may not be the same as the water-soluble polymer used to form the dried coating of water-soluble polymer. Additional solid excipients may mix with the water-soluble polymer to form the first solid phase. Alternatively, additional solid excipients may phase separate from the first solid phase comprising water-soluble polymer to form a second, or additional, solid phase(s).

In one embodiment, the coating solution will preferably spread relatively uniformly across the array. Such spreading will desirably lead to a relatively uniform application of coated material to the microneedle array. That is, the amount of coated material at and near the edges of the array will be similar to the amount of coated material at or near the center of the array. Alternatively, the surface properties of the coating solution may be adjusted to control the amount of spreading of the coating solution, thereby allowing for application of controlled amounts of coating material at specified locations on the microneedle array. Removal of the carrier fluid is typically accomplished by evaporation, which may be allowed to take place at ambient conditions or may be adjusted by altering the temperature or pressure of the atmosphere surrounding the microneedle array. Evaporation conditions are desirably selected so as to avoid degradation of the coating material. In certain embodiments, the active agent may preferentially adsorb onto the dried polymer coating prior to complete removal of the carrier fluid, as described in U.S. patent application Ser. No. 60/754786, “Methods for Coating Microneedles”, filed on Dec. 29, 2005, the disclosure of which is herein incorporated by reference.

In some embodiments removal of all or substantially all of the carrier fluid is desired. It should be understood, however, that relatively small amounts of carrier fluid may remain in the resultant matrix coating. For example, where the carrier fluid comprises water, the resultant matrix coating may typically contain between about 0.1 to 30% by weight of water, often between about 1% to 20% by weight of water, and sometimes between about 1% and 10% by weight of water.

Although not wishing to be bound by theory, it is believed that in certain embodiments the following mechanism allows for formation of the aforementioned multi-phase matrix coating having a continuous, first solid phase. The dried coating of water-soluble polymer allows for a hydrophilic coating solution to wet out most or all of the surface of the microneedle array. The coating solution then dissolves a portion of the water-soluble polymer, while simultaneously evaporating. A thin layer of water-soluble polymer in intimate contact with the microneedle array is not dissolved, and thus prevents or minimizes interaction of active substance with the microneedle array surface. The water-soluble polymer that dissolves into the coating solution subsequently phase separates when the carrier fluid is removed (e.g., by evaporation) from the coating solution. Once the carrier fluid is removed, the water-soluble polymer forms a porous structure that is filled with non-volatile components of the coating solution. The non-volatile components of the coating solution may include materials that form a liquid phase, as well as materials that may form a second solid phase within the pores of the first solid phase formed by the water-soluble polymer. Thus several benefits are achieved. The non-volatile components of the coating solution (e.g., active substance, etc.) are protected from contact, and thus possible irreversible adhesion, with the microneedle surface. The non-volatile components of the coating solution are retained on the surface of the microneedle array by the porous polymer structure, thus minimizing the potential that active substance can be removed accidentally from the surface. The non-volatile components of the coating solution, and in particular the active substance(s), however, are not intimately mixed with the water-soluble polymer. As such, they do not need to slowly diffuse from the polymer-rich phase of the matrix and are thus relatively easily removed from the matrix when the matrix is brought into contact with a hydrophilic medium, such as the interstitial fluid in a skin layer. It should be understood, however, that some of the non-volatile components may mix with the water-soluble polymer and that some or all of the water-soluble polymer may be delivered from the array in other embodiments.

In another embodiment, a masking fluid may be applied to the microneedle array prior to application of the coating solution as described in International Patent Application Publication No. WO 06/055799, the disclosure of which is herein incorporated by reference. For example, a masking fluid, such as a hydrofluoroether, may be applied to partially cover the microneedle array and then the coating solution may be applied to the surface of the masking fluid. In some embodiments, use of a masking fluid may help to control the location of matrix coating ultimately left on the microneedle array.

One or more surfaces of the microneedle arrays may be altered with a surface pre-treatment prior to application of the water-soluble polymer. Typical surface pre-treatments include a variety of plasma treatments capable of altering surface functionality. For example, polycarbonate may be plasma treated with a nitrogen plasma to cause amide functionalization or with an oxygen plasma to cause carboxylate functionalization. A combination of nitrogen and oxygen plasma treatment may be used to give a mixed surface functionality.

In one embodiment, different portions of the coating material may be preferentially deposited in different locations on the microneedle array. For example, where the coating material comprises a pharmaceutically effective substance (such as an antigen), it may be desirable to preferentially deposit the pharmaceutically effective substance on or near the tips of the microneedles. In some cases more than 30%, sometimes more than 50%, and occasionally more than 75% of the pharmaceutically effective substance by weight is deposited on the microneedles. In some cases the pharmaceutically effective substance preferentially resides on the upper half of the microneedles, that is, the portion of the microneedles away from the substrate. In one embodiment substantially no pharmaceutically effective substance is deposited on the substrate, that is, substantially all of the pharmaceutically effective substance is deposited on the microneedles. In one embodiment, substantially all of the pharmaceutically effective substance is deposited on the upper half of the microneedles. By substantially all, it should be understood that insignificant amounts of pharmaceutically effective substance, for example less than about 5% by weight, preferably less than about 1% by weight of the pharmaceutically effective substance is not deposited on the upper half of the microneedles. The total amount of material deposited on the microneedle array (i.e., water-soluble polymer, surfactant, other excipients, etc.) may be distributed differently from the distribution of active substance. For example, more than 50% by weight of the total amount of material deposited may be deposited on the substrate of the array.

In any of the foregoing embodiments, any number of conventional coating methods may be used to apply the coating solution to the microneedle array including dipping, brushing, drop coating, precision volumetric dispensing, gravure coating, and spray coating. In one embodiment, the coating solution may be applied as a metered amount of one or more droplets that are allowed to spread across the array substrate.

In one embodiment, a microneedle array may be applied to a skin surface in the form of a patch, such as described in International Patent Application Publication WO 05/123173, the disclosure of which is herein incorporated by reference. A microneedle device may comprise a patch in the form of a combination of an array, a pressure sensitive adhesive and backing. Microneedles may protrude from all or a portion of the microneedle array substrate surface. The microneedles may be arranged in any desired pattern, such as uniformly spaced rows, or distributed over the microneedle substrate surface randomly. In one embodiment, arrays of the present invention have a distal-facing surface area of more than about 0.1 cm² and less than about 20 cm², preferably more than about 0.5 cm² and less than about 5 cm². In one embodiment, a portion of the substrate surface of the patch is non-patterned. In one embodiment the non-patterned surface has an area of more than about 1 percent and less than about 75 percent of the total area of the device surface that faces a skin surface of a patient. In one embodiment the non-patterned surface has an area of more than about 0,10 square inch (0.65 cm²) to less than about 1 square inch (6.5 cm²). In another embodiment, the microneedles are disposed over substantially the entire surface area of the array.

Microneedle devices may be used for immediate delivery, that is where they are applied and immediately removed from the application site, or they may be left in place for an extended time, which may range from a few minutes to as long as 1 week. In one aspect, an extended time of delivery may be from 1 to 30 minutes to allow for more complete delivery of a drug than can be obtained upon application and immediate removal. In another aspect, an extended time of delivery may be from 4 hours to 1 week to provide for a sustained release of drug.

### EXAMPLES

The following examples are presented merely to further illustrate features, advantages, and other details of the invention. It is to be expressly understood, however, that while the examples serve this purpose, the particular materials and amounts used as well as other conditions and details are not to be construed in a matter that would unduly limit the scope of this invention.

### Tetanus Toxoid Total-Array Content by High Performance Liquid Chromatography (HPLC)

A sample extraction solvent was prepared containing 50 mM potassium perchlorate, 50 mM potassium citrate, 20 mM sodium phosphate, 376 mM sodium chloride, and 100 μg/mL bovine serum albumin. An HPLC sample solution was prepared by placing an array into a polypropylene cup, adding 1.0 mL of the sample extraction solvent to the cup, snapping a cap onto the sample cup, and sonicating for 30 minutes. Gradient elution HPLC (Mobile phase A): 0.2% (v/v) perchloric acid; Mobile phase B: 10% water, 88% acetonitrile, 2% isopropanol, 0.2% perchloric acid (70%); Solvent Program:_{—}0.00 min, 22% B, 1.0 mL/min; 6.00 min, 58% B, 1.0 mL/min; 6.01 min, 100% B, 1.0 mL/min; 6.50 min, 100% B, 0.5 mL/min; 10.0 min, 0% B, 0.5 mL/min; Injection Volume: 100 μL; Column: Zorbax 300SB-C8 50×4.6mm, 3.5 micron) was used to quantify tetanus toxoid in the HPLC sample solution. Non-adjuvanted tetanus toxoid (TT) vaccine (Aventis) was calibrated against a lyophilized TT primary standard (List Biologics) and used as a working standard. The working standard was used to obtain a calibration curve from approximately 1 μg-TT/mL to 28 μg-TT/mL. The correlation coefficient for the linear regression of the calibration curve was typically greater than 0.999. Tetanus toxoid content results are the average of between 6 and 10 replicates.

### Tetanus Toxoid Tip-Content by High Performance Liquid Chromatography (HPLC)

Tetanus toxoid content on the tips of the microneedles was measured by fixing the toxoid in place on the substrate and lower portions of the microneedles so that it could not be extracted into the HPLC sample solution. A microneedle array was placed on a flat surface with the needles pointing upward and 10 μL of an oil-based polyurethane coating solution (Minwax Fast-Drying Polyurethane) was applied to the array and allowed to coat the substrate of the array. The polyurethane was allowed to cure for at least 3 hours at ambient conditions. The array was subsequently extracted and analyzed as described in the total content method.

### Microneedle Arrays

Microneedle arrays were prepared as follows. A circular disk (area 2 cm², thickness 1.02 mm) that was partially patterned with an array of microneedles (37×37) in a square shape (1 cm²) centered on one side of the disk was prepared. The needles were regularly spaced with a distance of 275 microns between the tips of adjacent needles in a square-shaped pattern. Individual needles were pyramidal in shape with a height of 250 microns and a square base having a side-length of 83.3 microns. The tips were truncated with a flat, square-shaped top having a side-length of 5 microns. Arrays were injection molded according to the general description provided in International Patent Application Publication No. WO 05/82596 and made from polycarbonate (LEXAN HPS1R-1125, GE Plastics, Pittsfield, Mass.). The center of the disk was then die cut to provide a microneedle array (area=1 cm²) having microneedles on approximately 90% of the surface of the patterned side of the disk. The microneedle array had approximately 1200 microneedles,

### Example 1

A polyvinylpyrrolidone (PVP) stock solution was prepared by adding 825 mg PVP (Plasdone K-29/32, Povidone USP, ISP Technologies, Wayne, N.J.) to 25 mL water and mixing until the PVP was dissolved. A stock solution was prepared by adding 50 mg polysorbate 80 (TWEEN 80, Sigma Chemical Co., St. Louis, Mo.) to 25 mL ethanol. A diluted stock solution was prepared by adding 2 mL of the polysorbate stock solution to 18 mL ethanol. A PVP coating solution was prepared by adding 1 mL of the PVP stock solution to 9 mL of the diluted polysorbate stock solution. A microneedle array was placed on a flat surface with the needles pointing upward and an aliquot of 30 μL of the PVP coating solution was applied to the center of the array using a pipette and allowed to spread across the array. The PVP coating solution was allowed to dry at ambient conditions.

TWEEN −80 (90 mg) was added to water (30 mL) to prepare a TWEEN −80 stock solution with a concentration of 3 mg/mL. PVP (1.8 g) was added to water (20 mL) to prepare a PVP stock solution with a concentration of 90 mg/mL. Sucrose (1.8 g) was added to water (20 mL) to prepare a sucrose stock solution with a concentration of 90 mg/mL. Potassium citrate (1.8 g) was added to water (20 mL) to prepare a potassium citrate stock solution with a concentration of 90 mg/mL. An antigen coating formulation was prepared by mixing tetanus toxoid (Statens Serum Institute Lot 92-1, 888 Lf/mL) with aliquots of the TWEEN −80, PVP, sucrose and potassium citrate stock solutions.

An aliquot (15 μL) of masking fluid (FC-43 FLUORINERT Electronic Liquid) was applied to the center of the array using a pipette and allowed to spread across the array. A 10 μL aliquot of the antigen coating formulation was applied to the center of the masking fluid on the array using a pipette. The nominal amount of tetanus toxoid in the applied antigen coating formulation was 10 μg. The nominal amount of TWEEN −80 in the applied antigen coating formulation was 6 μg. The nominal amounts of PVP, sucrose, and potassium citrate were 100 μg. The volatile components of the antigen coating formulation and the masking fluid were allowed to evaporate at ambient conditions for approximately 30 minutes to provide an antigen-containing coating on the array. Tetanus toxoid total-array content as measured by reversed phase HPLC was 11.9 μg (st. dev.=0.5 μg). Tetanus toxoid tip-content was measured as 5.0 μg (st. dev.-1.2 μg).

### Examples 2-5

Coated arrays were prepared according to the procedure described in Example 1 with the exception that the nominal amounts of PVP, sucrose and potassium citrate were varied, as shown in Table 1. Tetanus toxoid content of the coated array as measured by reversed phase HPLC and tetanus toxoid content on the tips of the microneedles was measured. The results are shown in Table 1.

TABLE 1

| | | | | Tetanus toxoid content | |
|---|---|---|---|---|---|
| | | | Potassium | Total-array, | |
| Ex. | PVP | Sucrose | citrate | Mean (st. dev) | Tip-content, |
| No. | [μg] | [μg] | [μg] | [μg] | Mean (st. dev) [μg] |
| 1 | 100 | 100 | 100 | 11.9 (0.5) | 5.0 (1.2) |
| 2 | 100 | 100 | 10 | 13.1 (0.4) | 8.5 (0.5) |
| 3 | 10 | 10 | 100 | 11.6 (0.8) | 9.3 (1.3) |
| 4 | 10 | 100 | 10 | 11.5 (0.3) | 9.1 (1.4) |
| 5 | 100 | 10 | 10 | 12.3 (0.3) | 6.9 (0.7) |

### In Vivo Tetanus Toxoid Deposition

Microneedle devices were prepared by adhering antigen coated arrays as described in Examples 1 to 5 to an adhesive backing. The arrays were applied to hairless guinea pigs using an applicator as generally described in U.S. Patent Application Ser. No. 60/578,651, the disclosure of which is hereby incorporated by reference. The applicator piston mass was 5.08 g and the devices were applied at a velocity of 8.07 meters/second. The devices were applied to sites on the soft tissue of the abdomen and muscle on the lower back below the ribs and just above the pelvis. The application sites were cleaned with 70% isopropyl alcohol and allowed to air dry for at least 30 seconds prior to device application. Devices (N=5) were removed at specified time points and the tetanus toxoid content remaining on the arrays was measured by HPLC. The results are summarized in Table 2.

TABLE 2

| | Tetanus toxoid content [μg] | | | | |
|---|---|---|---|---|---|
| Array | T = 0 | | | | |
| Example No. | min | T = 1 min | T = 5 min | T = 10 min | T = 20 min |
| 1 | 11.9 | 9.8 | 10.3 | 8.6 | 8.1 |
| 2 | 13.1 | 10.9 | 10.1 | 8.2 | 8.1 |
| 3 | 11.6 | 9.1 | 8.1 | 7.6 | 6.3 |
| 4 | 11.5 | 9.9 | 8.6 | 8.2 | 7.0 |
| 5 | 12.3 | 11.1 | 9.8 | 10.0 | 8.5 |

### Example 6

A polyvinyl alcohol coating solution was prepared as follows. An amount (250 mg) of polyvinyl alcohol (80% hydrolyzed, typical MW=9,000-10,000, CAS 9002-89-5, Aldrich, St. Louis, Mo.) was added to water (25 mL) to prepare a polyvinyl alcohol stock solution. An aliquot of polyvinyl alcohol stock solution (2 mL) was added to ethanol (18 mL) to prepare a polyvinyl alcohol coating solution. A microneedle array was placed on a flat surface with the needles pointing upward and an aliquot of 30 μL of the polyvinyl alcohol coating solution was applied to the center of the array using a pipette and allowed to spread across the array. The polyvinyl alcohol coating solution was allowed to dry at ambient conditions. An aliquot (15 μL) of masking fluid (FC-43 FLUORINERT Electronic Liquid) was then applied to the center of the array using a pipette and allowed to spread across the array. A 10 μL aliquot of the antigen coating formulation was applied to the center of the masking fluid on the array using a pipette. Antigen coating formulations were prepared according to the general procedure described in Example 1. The nominal amount of tetanus toxoid in the applied antigen coating formulation was 10 μg. The nominal amount of TWEEN-80 in the applied antigen coating formulation was 6 μg. The nominal amounts of PVP, sucrose, and potassium citrate were 100 μg. The volatile components of the antigen coating formulation and the masking fluid were allowed to evaporate at ambient conditions for approximately 30 minutes to provide an antigen-containing coating on the array. Tetanus toxoid total-array content as measured by reversed phase HPLC was 10.4 μg (st. dev.=0.7 μg). Tetanus toxoid tip-content was measured as 9.3 μg (st. dev.=0.4 μg).

### Examples 7-14

Coated arrays were prepared according to the procedure described in Example 6 with the exception that the nominal amounts of PVP, sucrose and potassium citrate were varied, as shown in Table 3. Tetanus toxoid content of the coated array as measured by reversed phase HPLC and tetanus toxoid content on the tips of the microneedles was measured. The results are shown in Table 3.

TABLE 3

| | | | | Tetanus toxoid content | |
|---|---|---|---|---|---|
| | | | Potassium | Total-array, | Tip-content, |
| Ex. | PVP | Sucrose | citrate | Mean (st. dev) | Mean (st. dev) |
| No. | [μg] | [μg] | [μg] | [μg] | [μg] |
| 6 | 100 | 100 | 100 | 10.4 (0.7) | 9.3 (0.4) |
| 7 | 100 | 100 | 10 | 10.4 (0.3) | 8.2 (0.8) |
| 8 | 100 | 10 | 100 | 10.2 (0.6) | 9.1 (0.3) |
| 9 | 10 | 100 | 100 | 9.5 (0.9) | 6.7 (1.0) |
| 10 | 10 | 10 | 100 | 9.3 (0.4) | 5.8 (1.1) |
| 11 | 10 | 100 | 10 | 9.5 (0.5) | 6.9 (0.9) |
| 12 | 100 | 10 | 10 | 10.2 (0.4) | 5.6 (1.4) |
| 13 | 10 | 10 | 10 | 7.9 (0.2) | 4.5 (0.7) |
| 14 | 55 | 55 | 55 | 10.6 (0.4) | 8.4 (0.5) |

### Example 15

A coated array was prepared according to the procedure described in Example 7. Tetanus toxoid total-array content as measured by reversed phase HPLC was 10.7 μg (st. dev.=0.9 μg). Tetanus toxoid tip-content was measured as 8.7 μg (st. dev.=0.6 μg). Arrays were applied to hairless guinea pigs as described above in the section “in vivo tetanus toxoid deposition”. The amount of tetanus toxoid remaining on the array after removal from the hairless guinea pig was measured by HPLC. The results are summarized in Table 4.

### Example 16

A coated array was prepared according to the procedure described in Example 8. Tetanus toxoid total-array content as measured by reversed phase HPLC was 11.4 μg (st. dev.=0.3 μg). Tetanus toxoid tip-content was measured as 8.6 μg (st. dev.=0.5 μg). Arrays were applied to hairless guinea pigs as described above in the section “in vivo tetanus toxoid deposition”. The amount of tetanus toxoid remaining on the array after removal from the hairless guinea pig was measured by HPLC. The results are summarized in Table 4.

### Example 17

A coated array was prepared according to the procedure described in Example 9. Tetanus toxoid total-array content as measured by reversed phase HPLC was 10.8 μg (st. dev.=0.3 μg). Tetanus toxoid tip-content was measured as 6.8 μg (st. dev.=0.9 μg). Arrays were applied to hairless guinea pigs as described above in the section “in vivo tetanus toxoid deposition”. The amount of tetanus toxoid remaining on the array after removal from the hairless guinea pig was measured by HPLC. The results are summarized in Table 4.

### Example 18

A coated array was prepared according to the procedure described in Example 13. Tetanus toxoid total-array content as measured by reversed phase HPLC was 11.7 μg (st. dev.=0.3 μg). Tetanus toxoid tip-content was measured as 5.3 μg (st. dev.=1.0 μg). Arrays were applied to hairless guinea pigs as described above in the section “in vivo tetanus toxoid deposition”. The amount of tetanus toxoid remaining on the array after removal from the hairless guinea pig was measured by HPLC. The results are summarized in Table 4.

TABLE 4

| | tetanus toxoid content [μg] | | | | |
|---|---|---|---|---|---|
| Array | T = 0 | | | | |
| Example No. | min | T = 1 min | T = 5 min | T = 10 min | T = 20 min |
| 15 | 10.7 | 10.6 | 8.8 | 7.8 | 6.3 |
| 16 | 11.4 | 10.2 | 8.4 | 8.1 | 7.3 |
| 17 | 10.8 | 9.3 | 9.2 | 8.4 | 7.5 |
| 18 | 11.7 | 9.7 | 9.7 | 8.3 | 7.9 |

### Example 19

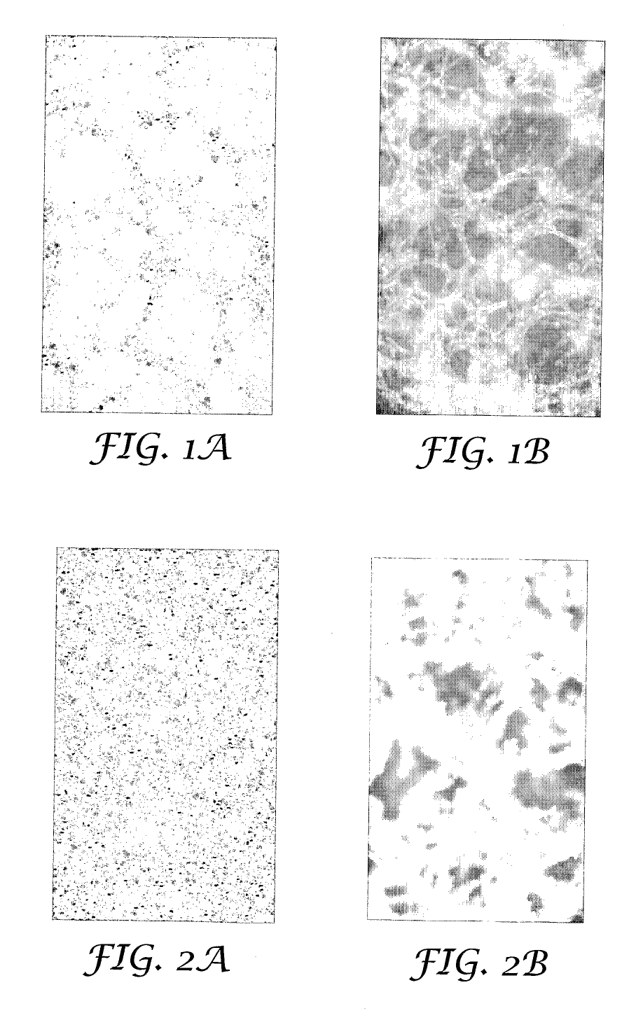

A glass substrate (50 mm×75 mm microscope slide) was treated with a Basic Plasma Cleaner (available from Harrick Scientific, Ithaca, N.Y.) for 2 minutes. A 0.3 wt-% solution of poly(vinyl alcohol) in water was prepared using 85-89% hydrolyzed poly(vinyl alcohol) having a 4% solution viscosity of 100 cP (available from Spectrum Chemical Manufacturing Co., Gardena, Calif. as catalog no. P1180). An aliquot of 200 μL of the poly(vinyl alcohol) solution was spread over a 20 cm² area of the glass substrate and allowed to dry overnight at ambient conditions. A placebo coating solution was prepared having a concentration of 2.3 wt-% polyoxyethylene (20) sorbitan monooleate (available from Uniqema, New Castle, Del. as TWEEN-80) in water. An aliquot of 200 μL of the coating solution was applied to the 20 cm² area of the glass substrate covered by the dried poly(vinyl alcohol). The glass substrate was then placed in an oven for 30 minutes at 45° C. to allow the water to evaporate. A photograph of the multi-phase matrix coating (using a Nikon Eclipse ME600 microscope with a 40× objective and a Nikon DXM 1200F digital camera with a 10× ocular) is shown in FIG. 1A. The multi-phase matrix coating was subsequently washed with an ethyl acetate/heptane mixture (1:1 by weight) to remove the polyoxyethylene (20) sorbitan monooleate. The washed sample was then dried to remove the ethyl acetate/heptane mixture. A photograph of the remaining porous solid coating, using the same conditions as above, is shown in FIG. 1B.

### Example 20

A sample was prepared as described in Example 19, with the exception that the coating solution had a concentration of 1.84 wt-% polyoxyethylene (20) sorbitan monooleate and 0.46% sucrose in water. A photograph of the multi-phase matrix coating is shown in FIG. 2A and the remaining porous solid coating (after washing) is shown in FIG. 2B.

### Example 21

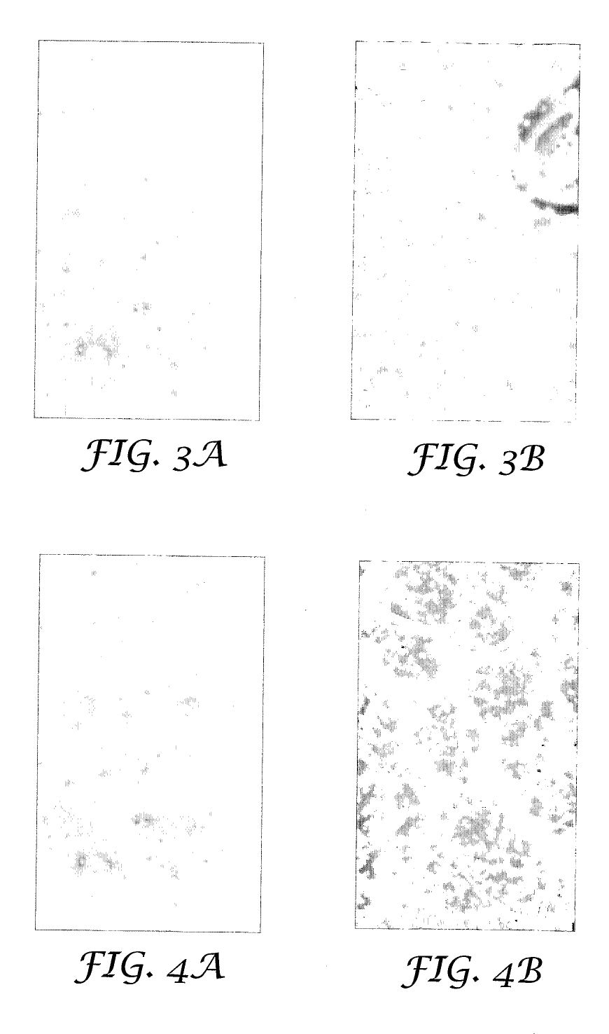

A sample was prepared as described in Example 19, with the exception that the coating solution had a concentration of 2.07 wt-% polyoxyethylene (20) sorbitan monooleate and 0.23% sucrose in water. A photograph of the multi-phase matrix coating is shown in FIG. 3A and the remaining porous solid coating (after washing) is shown in FIG. 3B.

### Example 22

A sample was prepared as described in Example 20, with the exception that the coating solution contained trehalose instead of sucrose. A photograph of the multi-phase matrix coating is shown in FIG. 4A and the remaining porous solid coating (after washing) is shown in FIG. 4B.

### Example 23

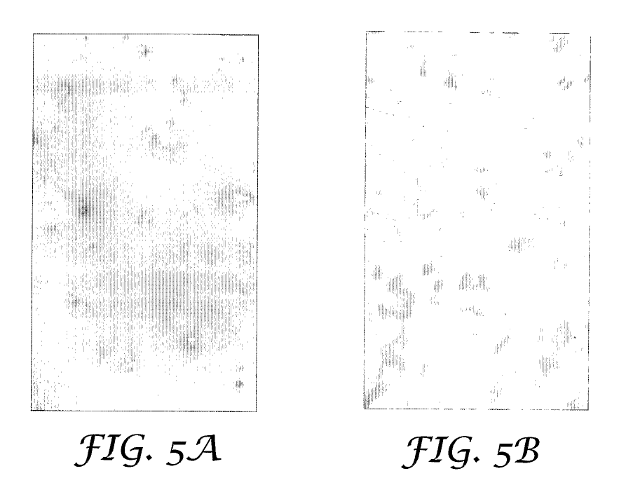

A sample was prepared as described in Example 21, with the exception that the coating solution contained trehalose instead of sucrose. A photograph of the multi-phase matrix coating is shown in FIG. 5A and the remaining porous solid coating (after washing) is shown in FIG. 5B.

### Example 24

A poly (vinyl alcohol) (PVA) stock solution was prepared by adding 72 mg of PVA (MW 9,000-10,000, 80% hydrolyzed, Aldrich, Milwaukee, Wis.) to 5 mL of diluent (12 mg/mL sucrose in phosphate buffered saline) and mixed until dissolved. A diluted PVA solution was prepared by adding 1 mL of the PVA stock solution to 9 mL of methanol. A microneedle array was placed on a flat surface with the needles pointing upward and an aliquot of 30 μL of the diluted PVA stock solution was applied to the center of the array using a pipette and allowed to spread across the array. The PVA solution was allowed to dry at ambient conditions. Antigen coating formulations were prepared by mixing tetanus toxoid (Statens Serum Institute Lot 92-1, 888 Lf/mL) with equal volumes of either PBS or PBS containing sucrose (12 mg/mL).

An aliquot (15 μL) of masking fluid (FC-43 FLUORINERT Electronic Liquid) was applied to the center of the array using a pipette and allowed to spread across the array. A 10 μL aliquot of the antigen coating formulations were applied to the center of the masking fluid on an array using a pipette. The nominal amount of tetanus toxoid in the applied antigen coating formulations was 18 μg. The nominal amount of sucrose applied in the sucrose containing antigen coating formulation was 60 μg. The volatile components of the antigen coating formulation and masking fluid were allowed to evaporate at ambient conditions for approximately 30 minutes to provide an antigen-containing coating on the arrays. Tetanus toxoid total-array content as measured by reversed phase HPLC for the PBS and PBS containing sucrose were 17.14 mcg (st.dev.=0.39 μg) and 17.24 μg (st.dev.=0.54 μg), respectively. Tetanus toxoid tip-content for the PBS and PBS containing sucrose were measured as 9.17 μg (st.dev.=1.61 μg) and 9.57 μg (st.dev.=2.99 μg), respectively.

We claim:

1. A drug delivery device comprising:
a microneedle array comprising a substrate and a plurality of microneedles extending out from the substrate; and
a multi-phase matrix coating containing an active substance on at least a portion of the microneedle surface;
wherein the multi-phase matrix coating comprises a first solid phase and a liquid phase, and
wherein the first solid phase comprises a water-soluble polymer.

2. A drug delivery device as claimed in claim 1 wherein the liquid phase comprises a surfactant.

3. A drug delivery device as claimed in claim 1 wherein the active substance is an antigen.

4. A drug delivery device as claimed in claim 1 wherein the liquid phase is continuous.

5. A drug delivery device as claimed in claim 1 wherein the first solid phase is continuous.

6. A drug delivery device as claimed in claim 1 wherein the water-soluble polymer is selected from the group consisting of polyvinyl alcohol, polyvinyl pyrrolidone, and polyethylene glycol.

7. A drug delivery device as claimed in claim 1 wherein the concentration of water-soluble polymer in the first solid phase is greater than about 50% by weight.

8. A drug delivery device as claimed in claim 1 wherein the multi-phase matrix coating further comprises a second solid phase.

9. A drug delivery device as claimed in claim 8 wherein the second solid phase comprises a sugar.

10. A drug delivery device as claimed in claim 1 wherein the substrate and the plurality of microneedles are made from a water-insoluble polymer.

11. A drug delivery device as claimed in claim 1 wherein the weight ratio of first solid phase to liquid phase is less than about 50:50.

12. A drug delivery device as claimed in claim 1 wherein the liquid phase has a viscosity of between about 0.1 and 5 Pa·s.

13. (canceled)

14. A method of preparing a drug delivery device comprising the steps of:
a) providing a microneedle array having a substrate and a plurality of microneedles extending out from the substrate;
b) applying a water-soluble polymer to the microneedle surface to form a dried coating of water-soluble polymer on at least a portion of the microneedles;
c) preparing a coating solution comprising an active substance, a liquid capable of phase separating from the water-soluble polymer, and a carrier fluid;
d) applying the coating solution to the dried coating of water-soluble polymer; and
e) removing at least a portion of the carrier fluid from the array, thereby providing an active substance-containing matrix coating on the microneedle array.

15. A method as claimed in claim 14 and further comprising the step of: allowing the coating solution to remain in contact with the dried coating of water-soluble polymer for a length of time sufficient to allow at least a portion of the dried coating of water-soluble polymer to be dissolved into the carrier fluid.

16. A method as claimed in claim 14 wherein the carrier fluid is removed from the array by evaporation.

17. A method as claimed in claim 14 wherein the coating solution further comprises a sugar.

18. A method as claimed in claim 14 wherein the active substance-containing matrix coating has multiple phases.

19. A method as claimed in claim 18 wherein the active substance-containing matrix has a first solid phase and a liquid phase.

20. A method as claimed in claim 14 wherein the liquid capable of phase separating from the water-soluble polymer is a surfactant.

21. A method as claimed in claim 14 wherein a surfactant-containing aqueous solution is used to apply the water-soluble polymer to the microneedle surface to form a dried coating of water-soluble polymer.
